(19) United States
(12) Patent Application Publication
(10) Pub. No.: US 2006/0122504 A1
(43) Pub. Date: Jun. 8, 2006

(54) Electronic subsystem with communication links

(76) Inventors: Thaddeus John Gabara, Murray Hill, NJ (US); Vladimir Prodanov, New Providence, NJ (US)

Correspondence Address: Michael J. Urbano, 1445 Princeton Drive, Bethlehem, PA, 18017-9166, US

(21) Appl. No.: 10/993,532

(22) Filed: Nov. 19, 2004

(51) Int. Cl. A61B 8/00

(52) U.S. Cl. 600/437

(57) ABSTRACT

Electronic modules are interconnected with one another by means of communication (e.g., ultrasonic) links, In one embodiment, in a local conference call environment, only one wireless RF link is necessary—between a master cell phone and a base station, whereas all other voice modules are interconnected with one another and with the master via ultrasonic links. In another embodiment, a master voice module (with or without an RF link to a base station) includes at least one detachable module (e.g., an earpiece and/or mouthpiece) that is interconnected with the master via an ultrasonic link. In yet another embodiment, a detachable module includes a capacitor, which serves as its power supply and which is recharged when it is attached a master module (e.g., by a battery in the master module).

### BACKGROUND OF THE INVENTION

1. Field of the Invention

This invention relates to electronic modules that are interconnected by communication links (e.g., free space, ultrasonic links) and to such modules that have detachable, rechargeable units.

2. Discussion of the Related Art

Wireless links between electronic modules are common. In the telecommunications industry wireless links interconnect cell phones and base stations, and in the computer industry wireless links interconnect PCs and their peripherals. Typically such links utilize RF to establish the connections. For example, in the telecommunications industry a local switching system may interconnect several cell phone users into a conference call using Bluetooth RF connectivity to provide local communications among the local cell phones. This type of system has several disadvantages: first, it wastes valuable wireless bandwidth to interconnect all users; second, the Bluetooth transceiver drains the cell phone battery since it needs to provide RF power.

Rechargeable, detachable electronic modules are also common. In the consumer industry products as diverse as flashlights and grass clippers include rechargeable batteries that are recharged when the detachable module is plugged into a master module, which is itself powered from an AC wall outlet. The use of a battery in the detachable module is too expensive for many of todays low cost, high-tech applications in which only low power ICs need to be powered.

### BRIEF SUMMARY OF THE INVENTION

In accordance with one aspect of our invention, electronic modules are interconnected with one another by means of free-space ultrasonic links, which require much less power than RF links but, of course, may have somewhat less range. In one embodiment, in a local conference call environment, only one wireless RF link is necessary—between a master cell phone and a base station, whereas all other modules are interconnected with one another and with the master via free-space ultrasonic links.

In accordance with another aspect of our invention, a master voice module (with or without an RF link to a base station) includes at least one detachable module (e.g., an earpiece and/or mouthpiece) that is interconnected with the master via an ultrasonic link.

In accordance with yet another aspect of our invention, the detachable module includes a capacitor, which serves as its power supply and which is recharged when it is attached to the master (e.g., by a battery in the master).

In accordance with still another aspect of our invention, first and second electronic modules are interconnected with one another by means of a communication link (e.g., electromagnetic, ultrasonic). The second module has a first state in which it is attached to the first module and a second state in which it is detached therefrom. The second module includes a low power VLSI circuit and a capacitor for providing electrical power thereto. The first module includes a battery for charging the capacitor when the second module is attached to the first module. This aspect of our invention is applicable, for example, to situations in which the second module includes a sensor (e.g., a gas or fingerprint sensor).

### BRIEF DESCRIPTION OF THE SEVERAL VIEWS OF THE DRAWING

Our invention, together with its various features and advantages, can be readily understood from the following more detailed description taken, in conjunction with the accompanying drawing, in which:

FIG. 1 is a block-diagrammatic view of an ultrasonic subsystem in accordance with the general principles of our invention;

FIG. 2 is a block-diagrammatic view of a pair of voice modules interconnected to one another by means of an ultrasonic link, in accordance with another embodiment of our invention;

FIG. 3 is a block-diagrammatic view of a voice module with a detachable earpiece/mouthpiece, in accordance with yet another embodiment of our invention;

FIG. 4 is a block-diagrammatic view of a cell phone with detachable modules and with an RF link to a base station, in accordance with one more embodiment of our invention;

FIG. 5 is a block-diagrammatic view of a voice module with two detachable earpieces for providing stereo reception, in accordance with still another embodiment of our invention;

FIG. 6 is a block-diagrammatic view of a cell phone conference call using ultrasonic links for local interconnect, in accordance with another embodiment of our invention; and

FIG. 7 is a block-diagrammatic view of a subsystem with a detachable module that includes a capacitor that powers a VLSI circuit within the detachable module and that is recharged when the detachable module is attached to a master module.

### DETAILED DESCRIPTION OF THE INVENTION

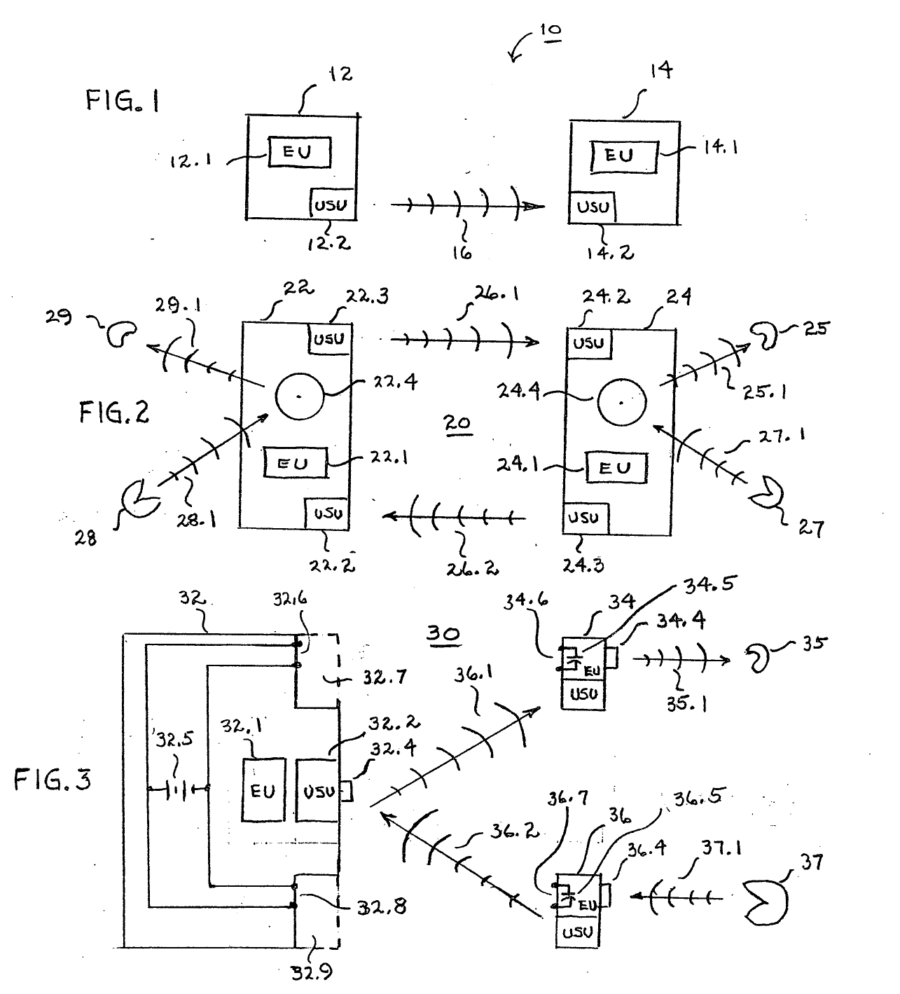

Turning now to FIG. 1, we show an electronic subsystem 10 including first and second electronic modules 12 and 14, respectively. Each module 12, 14 includes an electronic unit (EU) 12.1, 14.1, which controls its operation, and an ultrasonic unit (USU) 12.2, 14.2, which enables a free-space ultrasonic link to be established between the modules. Typically, the USUs each comprise ultrasonic transducers that convert electric signals into a free-space ultrasonic wave 16, which provides the communication link between the modules. The wave 16 may be encoded or modulated by the EU so as to convey information from one module to another. That information may take many forms: voice, data, or control signals, or any combination of them. For simplicity, in the illustration of FIG. 1 the ultrasonic wave 16 is shown to be directed from module 12 to module 14, but in practice it could also be directed from module 14 to module 12, or between both modules simultaneously or at different times.

Our ultrasound signals are generated at a frequency above the highest frequency that a human can hear; i.e., above about 20 kHz and illustratively in the range of about 25 kHz -1 Mhz.

In addition, it should be noted that the EUs and USUs may be identical units or they may be different from one another depending on the particular application.

In general, the subsystem 10 is primarily suited to short range links between modules; that is, depending on the power of the USUs, and the free-space atmospheric conditions between the modules, the likely effective range may be from approximately one foot to a few 10s of feet. As described in more detail below, the modules themselves may be designed to perform a variety of functions, and as such may be, for example, relatively complex cell phones, simpler voice communicators, detachable earpieces or mouthpieces, controllers coupled to appliances, telemetry apparatus, sensors, recorders, games etc.

One application, which is directed to a voice module local interconnect, is depicted in FIG. 2. Illustratively, the local interconnect subsystem 20 comprises at least two voice modules (e.g. cell phones) 22, 24, each of which includes, respectively, an EU 22.1, 24.1 a pair of USUs 22.2, 22.3 and 24.2, 24.3, and an audio transducer 22.4, 24.4 (e.g., a microphone, speaker, or both). Although the pair of USUs in each cell phone could be combined into a single USU for both transmission and reception (akin to a transceiver), we show them separately for sake of clarity. In particular, USUs 22.3, 24.3 are depicted as transmitters that generate (e.g., emit, encode) ultrasonic waves 26.1, 26.2, respectively, whereas USUs 22.2, 24.2 are depicted as receivers that receive (detect, decode) these waves. On the other hand the users of this subsystem are shown merely by the symbols of a mouth 27, 28, which generate audio voice waves 27.1, 28.1 that are received by transducers (i.e., microphones) 22.4, 24.4, respectively, and by the symbols of an ear 25, 29, which receive acoustic voice waves 25.1, 29.1 that are generated by transducers (i.e., speakers) 22.4, 24.4, respectively. Although only two voice modules are shown in the illustrative local interconnect subsystem 20, it will be readily appreciated by those skilled in the art that more than two voice modules can be interconnected in the same manner. On the other hand, it will also be appreciated that the two-module embodiment could also function as a walkie-talkie, but in this case the modules could be much simpler voice communicators rather than typically more complex and hence more expensive cell phones.

Advantageously, using ultrasonic links to interconnect modules consumes significantly less power than the RF links (or other forms of electromagnetic radiation) typical of the prior art.

Another application, which is directed to a voice module with a detachable unit (e.g., an earpiece or a mouthpiece), is depicted in FIG. 3. Illustratively, the subsystem 30 comprises a master voice module 32 and at least one detachable earpiece 34 or mouthpiece 36 (or both). The detachable units 34, 36 may be attached to the voice module 34 by any means well known in art (e.g., magnetic, mechanical). Illustratively, they are releasably snap fit into cavities 32.7, 32.8, respectively. The voice module comprises EU 32.1 and a USU 32.2 of the type described above, and in this case we have also depicted an ultrasonic transducer 32.4 that transmits/receives ultrasonic waves 36.1/36.2 to/from earpiece 34/mouthpiece 36. The latter include their own EUs, USUs as well as audio transducers 34.4, 36.4 (speakers, microphones), which transmit/receive audio to/from a human ear 35/mouth 37, respectively. Note, however, unlike the detachable mouthpiece 36, the detachable earpiece 34 merely decodes ultrasonic signal 36.1 (and does not have to generate one), it consumes considerably less power.

In one embodiment shown in FIG. 5, the subsystem 50 comprises a voice module 52 and two detachable modules both of which are earpieces 54, 56 that provide for stereo communication (e.g. to play MP3 files) to the user represented by the human ears 58, 59, respectively.

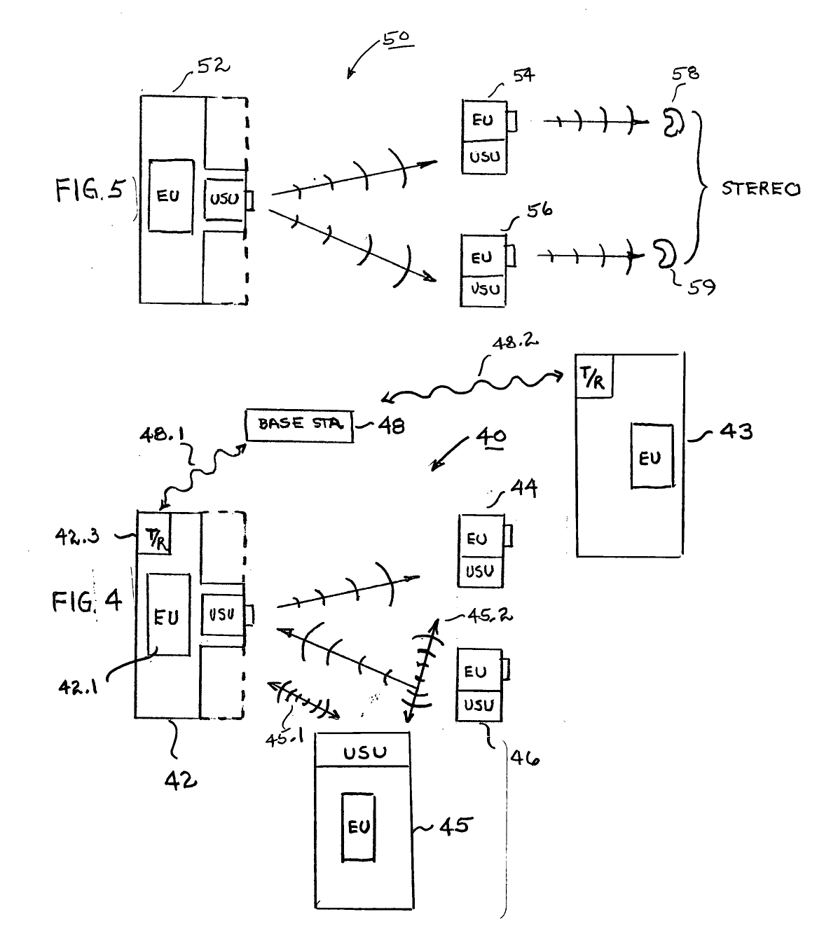

Another embodiment of our invention essentially provides the primary user of master voice module 32 to have handy a spare cell phone in the form of the detachable units. Thus, if a secondary user needs to be conferenced into a conversation between the primary user and a third party, then the primary user simply loans the detachable earpiece (for the secondary user to be able to listen in only) or both the detachable earpiece and mouthpiece (for the secondary user to be able to fully participate). The conferencing function may take on the form of a simple local interconnect as described with respect to FIG. 2, or it may include an RF link to a base station, as shown in FIG. 4. In the latter embodiment, the subsystem 40 comprises a master cell phone 42 and a pair of detachable modules (i.e., an earpiece 44 and a mouthpiece 46). In this case, however, the cell phone itself also includes an RF transceiver T/R 42.3, which for clarity is shown to be separate from EU 42.1, but could readily be a part of it. T/R 42.3 transmits/receives RF waves 48.1 that provide a communication link between cell phone 42 and base station 48. Conferencing with a remote user via another cell phone 43 would be established via base station using an RF link (RF waves 48.2), but conferencing with a local user via voice module 45 could be established via ultrasonic links (ultrasonic waves 45.1 and 45.2) in accordance with our invention or via a conventional RF link (in which case the voice module 45 would also be a cell phone).

It is to be understood that the above-described arrangements are merely illustrative of the many possible specific embodiments that can be devised to represent application of the principles of the invention. Numerous and varied other arrangements can be devised in accordance with these principles by those skilled in the art without departing from the spirit and scope of the invention. In particular, the various embodiments of our invention that involve multiple users (including the embodiments having detachable modules) may apply standard wireless transmission techniques to the ultrasonic signals, such as, for example frequency hopping or CDMA.

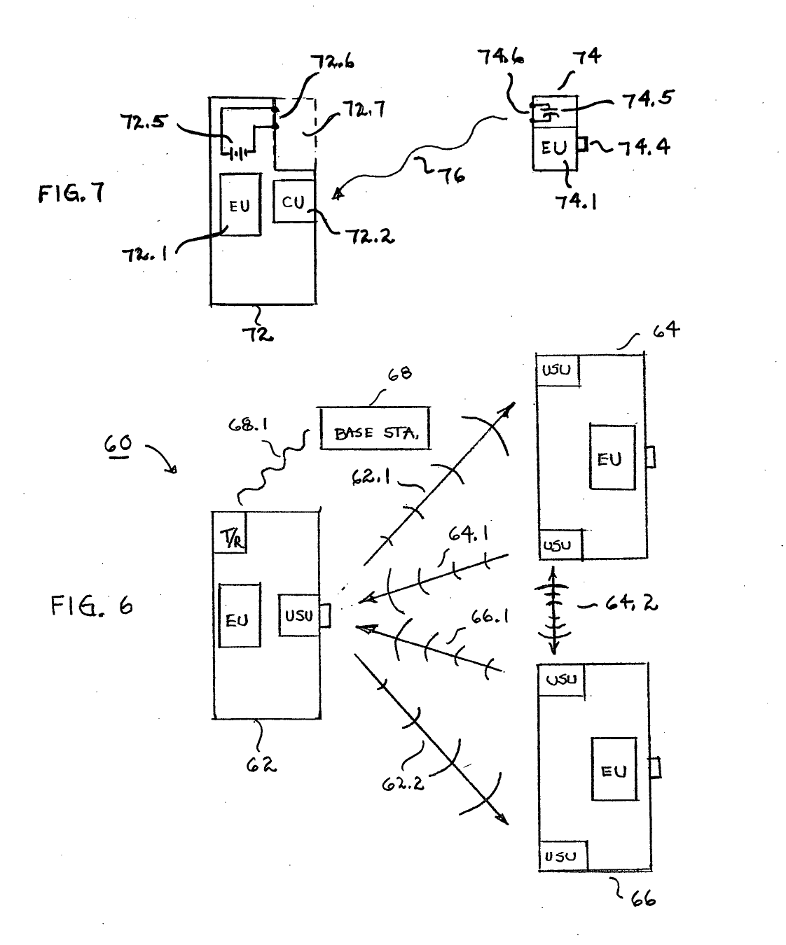

Moreover, it will be readily apparent to those skilled in the art that the conferencing arrangement depicted in FIG. 4 for a cell phone with detachable modules can extended to a similar arrangement for multiple cell phones and/or multiple voice modules, as shown in FIG. 6. Here, the subsystem comprises a master cell phone that has an established RF link (RF waves 68.1) to a remoter user (not shown) via base station 68. In addition, local user voice modules 64, 66 are conferenced with one another and with the master cell phone 62 via ultrasonic links (ultrasonic waves 62.1, 62.2, 64.1, 64.2, 66.1) in accordance with our invention.

In addition, with respect to the embodiments of our invention that include detachable modules, an optional feature provides for powering the detachable modules via an on-board capacitor, which is recharged when the module is attached to the voice module or cell phone. Thus, as illustrated in FIG. 3, the detachable earpiece/mouthpiece 34/36 has a capacitor 34.5/36.5, which powers the modules. These capacitors have external terminals 34.6/36.7, which make electrical contact with external terminals 32.6/32.8 located in cavity 32.7/32.9, respectively. Therefore, when the detachable modules are attached to the master module 32, the capacitors are recharged by means of a battery 32.5, which is located internal to the master module and is coupled to the terminals 32.6/32.8. An optional, but convenient feature that can be included in these embodiments is a sensor circuit within the EU to alert the user in advance that a detached module is running lower on power, so that the power capacitor of that module can recharged by attaching the module to the cell phone. Thus, for example, the subsystem might alert a user that a detached earpiece has five minutes of listening time left, so that the user can attach the earpiece to the cell phone to quickly recharge the power capacitor and then replace the earpiece back in the user's ear before the user stops talking.

We provide the following table to quantify the period the time that a detachable module can be detached from the powering module, assuming a single capacitor (size: 2 mm ×3 mm ×3 mm) continuously dissipating a constant amount of power indicated below, where Vᵢ and V_{f} are the initial and final voltage, respectively, and the activity rate is one third, which assumes that one-third of all gates in EU are switching constantly.

TABLE I
CONTINUOUS POWER DRAWN

| | CAPACITOR: 680 μF; | | |
|---|---|---|---|
| | Vᵢ = 1.1 V; V_{f} = 0.9 V | | |
| | 2 nW | 20 nW | 200 nW |
| Time | 37 hr | 3.7 hr | 0.37 hr |
| Boolean Operations | 107 × 10⁹ | 107 × 10⁹ | 107 × 10⁹ |
| Gates: 30,000 | 80 Hz | 803 Hz | 8 kHz |
| (active rate = 0.33) | | | |

Thus, under these circumstances the time that a detached module can remain detached without having to be recharged ranges from 37 hr to slightly more than a half hour. But, of course, if the detached module draws power for only a fraction of a duty cycle, then the length of time before recharging is increased accordingly, or the number of Boolean operations that can be performed is increased.

In Table II below our calculations reflect increasing the power supply (or initial) voltage from Vᵢ=1.1 V to 10 V to demonstrate how the number of Boolean operations can be increased by a factor of about 200 while keeping the charging time the same.

TABLE II
CONTINUOUS POWER DRAWN

| | DC-to-DC CONVERTER | | |
|---|---|---|---|
| | Vᵢ = 10 V; V_{f} = 3 V | | |
| | 372 nW | 3720 nW | 37200 nW |
| Time | 37 hr | 3.7 hr | 0.37 hr |
| Boolean Operations | 19.8 × 10¹² | 19.8 × 10¹² | 19.8 × 10¹² |
| Gates: 30,000 | 14.8 kHz | 148 kHz | 1.48 MHz |
| (active rate = 0.33) | | | |

The results of Table II assume that there are two powering capacitors and one inductor (i.e., in a standard DC-to-DC converter arrangement in the detachable module) in a MOSFET chip drawing constant power at V_{DD}=1 V. We also assume that the converter is running at a conservative efficiency rate of 80%.

The use of a detachable, capacitor-powered module is not, however, dependent on the use of an ultrasonic link between the detachable and master modules. Thus, as shown in FIG. 7, a master module 72 and a detachable, capacitor-powered module 74 have a communication link 76, which may be established via electromagnetic waves (e.g., RF waves, light waves) or via ultrasonic waves (as described above). Note, we include within the meaning of an electromagnetic link embodiments in which electrical contacts in the detachable module are brought into physical contact with electrical contacts in the master module. In general, the link may establish one-way (unidirectional) communication between the modules or two-way (bidirectional) communication between them.

In the embodiment illustrated in FIG. 7, the master module 72 includes an EU 72.1, which controls the operation of the module 72, and a communication unit (CU) 72.2, which generates and/or receives electromagnetic or ultrasonic waves. In addition, module 72 includes a battery 72.5 for recharging the detachable module 74 via external electrical contacts 72.6 when the detachable module 74 is plugged into the cavity 72.7 of the master module 72. (Note, these contacts 72.6, or separate ones, could also be used to establish a communication link with the corresponding contacts 74.6 of the detachable module 74.) On the other hand, the detachable module 74 includes an EU 74.1, which controls its operation, and a capacitor 74.5, which provides electrical power to EU 74.1. The capacitor 74.5 is recharged, as discussed above, via contacts 74.6 when the detachable module 74 is plugged into cavity 72.7 and contacts 74.6 and 72.6 physically touch one another.

Typically, EU 74.1 includes a VLSI circuit that requires very low electrical power (e.g., in the nanowatt-to-microwatt range as described in Tables I and II) to operate and hence can be powered by the charge stored in capacitor 74.5.

In an illustrative application, detachable module 74 includes a sensor 74.4 (e.g., a gas sensor or a fingerprint sensor) that detects a particular condition (e.g., a pollutant in the atmosphere; the pattern of a person's fingerprint). The EU 74.1 generates a signal that is encoded with data corresponding to the detected condition and transmits that signal to master module 72 via link 76 and CU 72.2.

The invention Of claim is:

1. An ultrasonic subsystem comprising:
a first electronic module including a first ultrasonic unit, and
a second electronic module including a second ultrasonic unit for communicating with said first unit via free-space ultrasonic waves.

2. The subsystem of claim 1, wherein said modules and each contain audio transducers, and said ultrasonic units communicate audio information from a transducer in one of said modules to a transducer in the other module.

3. The subsystem of claim 1, wherein said second module has a first state in which it is attached to said first module and a second state in which it is detached from said first module.

4. The subsystem of claim 3, wherein said second module is selected from the group consisting of at least one earpiece and at least one mouthpiece.

5. The subsystem of claim 4, wherein said second module includes a pair of earpieces that provide stereo broadcast therefrom.

6. The subsystem of claim 3, wherein said second module includes a capacitor for providing electrical power thereto, and said first module includes a battery for charging said capacitor when said second module is attached to said first module.

7. The subsystem of claim 1, wherein said first module includes an RF transceiver for communicating with a base station.

8. The subsystem of claim 7, wherein said first module comprises a cell phone.

9. The subsystem of claim 8, wherein said second module also comprises a cell phone.

10. A cellular subsystem comprising:
a first cell phone including an RF transceiver for communicating with a base station and a first ultrasonic unit,
an electronic module including a second ultrasonic unit for communicating with said first unit via free-space ultrasonic waves.

11. The subsystem of claim 10, wherein said module comprises a second cell phone.

12. The subsystem of claim 10, wherein said module has a first state in which it is attached to said cell phone and a second state in which it is detached from said cell phone.

13. The subsystem of claim 12, wherein said module is a component selected from the group consisting of at least one earpiece and at least one mouthpiece.

14. The subsystem of claim 13, wherein said module includes a pair of earpieces that provide stereo broadcast therefrom.

15. The subsystem of claim 12, wherein said module includes a capacitor for providing electrical power thereto, and said cell phone includes a battery for charging said capacitor when said module is attached to said cell phone.

16. A subsystem comprising
a first electronic module including a first communications unit, and
a second electronic module including a second communications unit for communicating with said first unit,
said second module has a first state in which it is attached to said first module and a second state in which it is detached from said first module,
said second module includes a capacitor for providing electrical power thereto, and said first module includes a battery for charging said capacitor when said second module is attached to said first module.

17. The subsystem of claim 16, wherein said second module further includes a sensor.

18. The subsystem of claim 16, wherein said communication link is selected from the group consisting of an electromagnetic wave link and an ultrasonic wave link.

19. The subsystem of claim 18, wherein said links are free-space links.

20. The subsystem of claim 18, wherein said electromagnetic link comprises electrical contacts in first and second modules, which contacts establish said link when said first module is attached to said second module.
